(19) United States
(12) Patent Application Publication
(10) Pub. No.: US 2014/0158898 A1
(43) Pub. Date: Jun. 12, 2014

(54) RADIATION IMAGE CAPTURING SYSTEM

(71) Applicant: KONICA MINOLTA, INC., Tokyo (JP)

(72) Inventor: Eiichi AKAGI, Tokyo (JP)

(73) Assignee: KONICA MINOLTA, INC., Tokyo (JP)

(21) Appl. No.: 14/095,715

(22) Filed: Dec. 3, 2013

(30) Foreign Application Priority Data
Dec. 11, 2012 (JP) 2012-269977

(51) Int. Cl. A61B 6/00

(52) U.S. Cl. CPC A61B 6/4208
USPC 250/394

(57) ABSTRACT

Disclosed is a radiation image capturing system. The radiation image capturing system includes a radiation image capturing apparatus and a capturing control apparatus. The radiation image capturing apparatus detects irradiating timing of radiation. The capturing control apparatus is able to communicate data to control the radiation image capturing apparatus. The capturing control apparatus includes, a storage section which stores capturing order information corresponded with a detecting condition of irradiating timing of radiation; an obtaining section which obtains capturing order information; and a first control section which reads the detecting condition corresponded to the obtained capturing order information and which transmits the read detecting condition to the radiation image capturing apparatus. The radiation image capturing apparatus includes a second control section which detects the irradiating timing based on the detecting condition.

### BACKGROUND

1. Field of the Invention

The present invention relates to a radiation image capturing system.

2. Description of Related Art

In the field of medicine, an FPD (Flat Panel Detector) is used as a radiation image capturing apparatus including a radiation detecting element which converts radiation such as an X-ray, etc. to an electric signal. The radiation generated by the radiation generating apparatus passes through a subject such as a human body and is irradiated on the FPD. After the radiation is irradiated, the charge accumulated in the radiation detecting element is read to generate an image. When the reading of the charge is not done swiftly, noise increases. Therefore, it is necessary to perform reading of the charge swiftly after the irradiation of the radiation.

As methods to detect timing of irradiation of radiation, there are an exposure signal linking method and an exposure signal non-linking method (for example, see Japanese Patent Application Laid-Open Publication No. 2011-254971). In the exposure signal linking method, the radiation generating apparatus and the radiation image capturing apparatus are connected with a signal line, and the exposure of radiation is detected by ON/OFF of the exposure signal output from the radiation generating apparatus. Turning to the exposure signal non-linking method, the radiation generating apparatus and the radiation image capturing apparatus are not connected with the signal line, and the radiation image capturing apparatus itself detects the exposure of radiation, such as by monitoring a portion or all of the radiation detecting elements included in the radiation image capturing apparatus for automatic detection of radiation.

However, with the exposure signal non-linking method, in other words, when the start of irradiation radiation is detected by the radiation image capturing apparatus itself, there is a possibility of error in detection due to a shock on the FPD panel or omission of detection due to the irradiated radiation being weak.

For example, when an image of a child is captured, the movement of the child may apply shock to the FPD panel, and this becomes a reason for error in detection. Moreover, when a stomach portion is captured, the radiation amount is low compared to other sites, the S/N ratio becomes low, and the possibility of omission of detection becomes high. Further, when the bone of the hand and finger is captured, the irradiating field is narrowed, radiation is hardly irradiated on the portion close to the outer circumference of the FPD panel, and the possibility of omission of detecting the radiation becomes high.

### SUMMARY

The present invention has been made in consideration of the above problems, and it is one of main objects to prevent error in detection and omission of detection when the irradiating timing of the radiation is detected by the radiation image capturing apparatus itself.

In order to achieve at least one of the above-described objects, according to an aspect of the present invention, there is provided a radiation image capturing system including:

a radiation image capturing apparatus which is provided with a plurality of radiation detecting elements to convert radiation to an electric signal, wherein the radiation image capturing apparatus detects irradiating timing of radiation by a radiation generating apparatus and generates an image based on the radiation which passes through a subject when the radiation is irradiated; and

a capturing control apparatus which is connected to the radiation image capturing apparatus to be able to communicate data and which controls the radiation image capturing apparatus,

wherein the capturing control apparatus includes,
a storage section which stores capturing order information corresponded with a detecting condition of irradiating timing of radiation by the radiation image capturing apparatus;
an obtaining section which obtains capturing order information regarding capturing by the radiation image capturing apparatus; and
a first control section which reads the detecting condition corresponded to the obtained capturing order information from the storage section and which transmits the read detecting condition to the radiation image capturing apparatus, and

the radiation image capturing apparatus includes,
a second control section which detects the irradiating timing of the radiation by the radiation generating apparatus based on the detecting condition transmitted from the capturing control apparatus.

According to the above aspect of the present invention, it is possible to prevent error in detection and omission of detection when detecting the irradiating timing of the radiation with the radiation image capturing apparatus itself.

Preferably, in the radiation image capturing system, the detecting condition includes sensitivity and/or region of the plurality of radiation detecting elements used when detecting the irradiating timing of the radiation.

Preferably, in the radiation image capturing system, the capturing order information includes a capturing site and/or a capturing direction.

Preferably, in the radiation image capturing system,

the capturing control apparatus includes,

a display section which displays a warning that the detecting condition is to be changed; and

an operation section to input an instruction to allow the detecting condition to be changed,

wherein when an instruction to allow the detecting condition to be changed is input on the operation section, the first control section transmits the detecting condition which is allowed to be changed to the radiation image capturing apparatus.

### BRIEF DESCRIPTION OF THE DRAWINGS

The present invention will become more fully understood from the detailed description given hereinbelow and the appended drawings, and thus are not intended to define the limits of the present invention, and wherein;

FIG. 1 is a diagram showing a system configuration of a radiation image capturing system of the present invention;

FIG. 2 is a block diagram showing a functional configuration of an FPD panel;

FIG. 3 is a diagram for describing a detecting method of irradiating timing of radiation;

FIG. 4 is a block diagram showing a functional configuration of a capturing control apparatus;

FIG. 5 is a diagram showing an example of a detecting condition correspondence table;

FIG. 6 is a diagram showing a detecting region in child chest and stomach portion simple capturing;

FIG. 7 is a diagram showing a detecting region in adult stomach portion front view capturing;

FIG. 8 is a diagram showing a detecting region in hand and finger bone front view capturing;

FIG. 9 is a ladder chart showing radiation image capturing processing in the FPD panel and the capturing control apparatus;

FIG. 10 is a ladder chart showing radiation image capturing processing in the FPD panel and the capturing control apparatus;

FIG. 11 is an example of a list screen;

FIG. 12 is an example of a capturing screen;

FIG. 13 is an example of a warning message screen to change the vibration error detection correction function to ON; and

FIG. 14 is an example of a warning message screen to change the detecting sensitivity to high sensitivity.

### DETAILED DESCRIPTION OF PREFERRED EMBODIMENTS

Below, an embodiment of the radiation image capturing system of the present invention is described with reference to the drawings. However, the present invention is not limited to the illustrated examples.

### [Configuration of Radiation Image Capturing System]

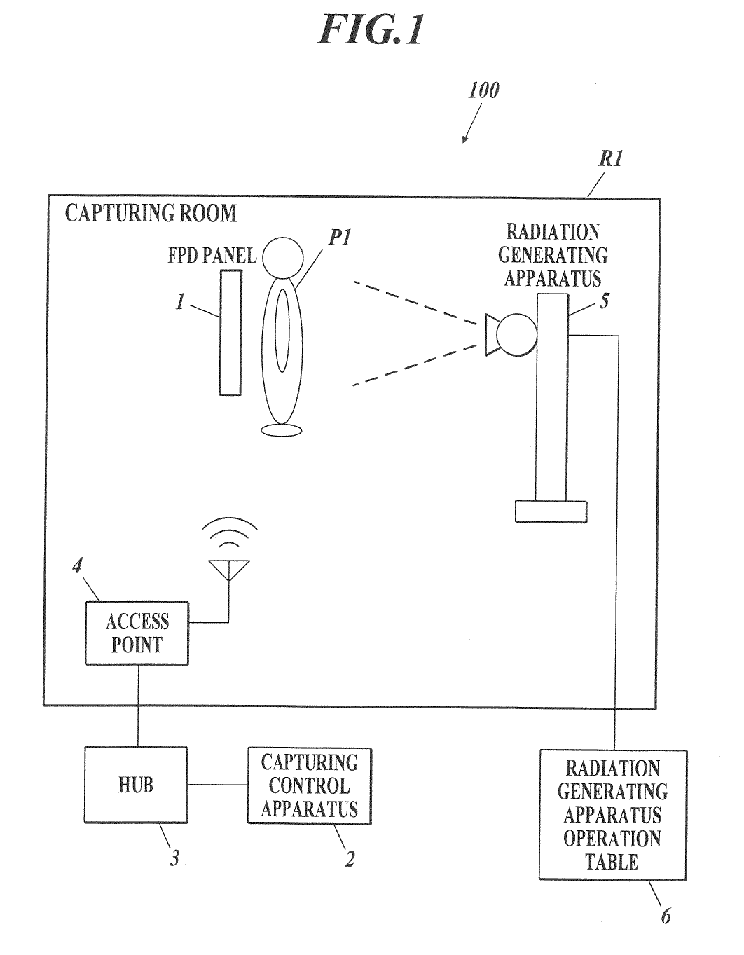

FIG. 1 shows a system configuration of a radiation image capturing system 100. The radiation image capturing system 100 includes an FPD panel 1, a capturing control apparatus 2, a hub 3, an access point 4, a radiation generating apparatus 5, a radiation generating apparatus operation table 6, and the like. The FPD panel 1, the access point 4, and the radiation generating apparatus 5 are provided in a capturing room R1 which blocks radiation. From the viewpoint of preventing exposure among patients, capturing is usually performed with one patient in the capturing room R1. The capturing control apparatus 2, the hub 3, and the radiation generating apparatus operation table 6 are provided outside the capturing room R1.

The FPD panel 1 is a planar cassette type radiation image capturing apparatus. The FPD panel 1 is irradiated from the radiation generating apparatus 5, and image data of a radiation image is generated based on the radiation which passes through a subject P1.

The capturing control apparatus 2 is an apparatus to control the FPD panel 1, and performs data communication with the FPD panel 1 by a wireless method through the hub 3 and the access point 4. In other words, the FPD panel 1 and the capturing control apparatus 2 are connected so as to be able to perform wireless data communication. The capturing control apparatus 2 obtains capturing order information from an RIS (Radiology Information System) and sets a condition for detecting irradiating timing of the radiation on the FPD panel 1 based on the obtained capturing order information. The capturing order information is information regarding a request for capturing such as a capturing target, capturing condition, and the like. The capturing order information includes, patient information, examination information, etc. The patient information is information regarding the patient, patient name, patient ID, birthday, sex, etc. The examination information is information regarding the examination including capturing site, capturing direction, etc.

The hub 3 is connected to the capturing control apparatus 2 and the access point 4, and relays data communication between the apparatuses.

The access point 4 includes an antenna to perform wireless data communication with the FPD panel 1.

The radiation generating apparatus 5 receives the radiation irradiating condition transmitted from the radiation generating apparatus operation table 6 and stores the received radiation irradiating condition. The radiation emitting condition includes a value of a tube current of a radiation tube, a value of a tube voltage, a filter type, radiation irradiating time, and the like. By pressing a switch to instruct irradiating timing provided on the radiation generating apparatus operation table 6, an instruction signal to irradiate radiation is transmitted to the radiation generating apparatus 5 from the radiation generating apparatus operation table 6, and the radiation generating apparatus 5 irradiates a predetermined amount of radiation for a predetermined amount of time based on the radiation irradiating condition.

The radiation generating apparatus operation table 6 obtains capturing order information from the RIS and transmits the radiation irradiating condition to the radiation generating apparatus 5 based on the obtained capturing order information. When the operator operates the radiation irradiating switch to instruct irradiating of the radiation, the radiation generating apparatus operation table 6 allows the radiation generating apparatus 5 to irradiate radiation to the subject P1 according to the set radiation irradiating condition.

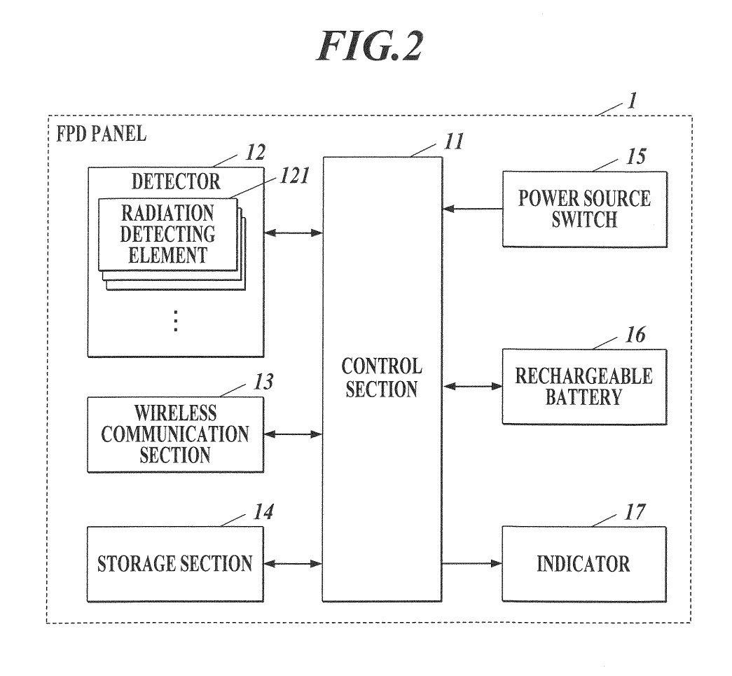

FIG. 2 shows a functional configuration of the FPD panel 1. As shown in FIG. 2, the FPD panel 1 includes a control section 11, a detector 12, a wireless communication section 13, a storage section 14, a power source switch 15, a rechargeable battery 16, an indicator 17, and the like.

The control section 11 is composed of a CPU (Central Processing Unit), a ROM (Read Only Memory), a RAM (Random Access Memory), and the like. The control section 11 reads a predetermined program stored in the ROM and expands the program in a work area of the RAM so that the CPU performs various processing according to the program. The control section 11 controls the detector 12, reads an image signal detected by the detector 12, generates image data of a radiation image based on the image signal, and transmits the generated image data to the capturing control apparatus 2 with the wireless communication section 13.

The detector 12 is composed of a plurality of radiation detecting elements 121 which converts the radiation to an electric signal, a scanning driving circuit which sends a pulse to the radiation detecting element 121 to scan/drive the radiation detecting element 121, and the like. The radiation detecting element 121 includes a photodiode (a photoelectric conversion element), etc. and is arranged two-dimensionally (matrix shape) on a radiation entering surface of the FPD panel 1.

The wireless communication section 13 includes an antenna apparatus which is not shown, and performs transmitting and receiving of data with the capturing control apparatus 2 through the access point 4 by a wireless method.

The storage section 14 includes a writable memory such as a flash memory and stores various pieces of data. For example, the storage section 14 stores image data generated based on the image signal detected by the detector 12. The storage section 14 stores the detecting condition of the irradiating timing of the radiation set on the capturing control apparatus 2. The detecting condition includes information such as detecting sensitivity, detecting region, and ON/OFF of vibration error detection correction of the plurality of radiation detecting elements 121 used in detecting the irradiating timing of the radiation.

The power source switch 15 is the switch for switching the power source of the FPD panel 1 between ON and OFF.

The rechargeable battery 16 supplies electric power to the functional sections of the FPD panel 1 based on the control of the control section 11.

The indicator 17 displays the state of the FPD panel 1, the remaining charge of the rechargeable battery 16 and the like.

The control section 11 detects the irradiating timing of the radiation by the radiation generating apparatus 5 based on the detecting condition of the irradiating timing of the radiation transmitted from the capturing control apparatus 2. In other words, the control section 11 functions as the second control section.

The control section 11 generates the image based on the radiation which passes through the subject P1 according to the irradiation of radiation. Specifically, when the control section 11 detects the irradiating timing of the radiation by the radiation generating apparatus 5, the control section 11 allows the radiation detecting elements 121 to start accumulating the charge for generating the image. After a predetermined amount of time passes, the control section 11 reads the charge accumulated in the radiation detecting elements 121, obtains the image signal based on the radiation amount which passes through the subject P1, and generates the image data.

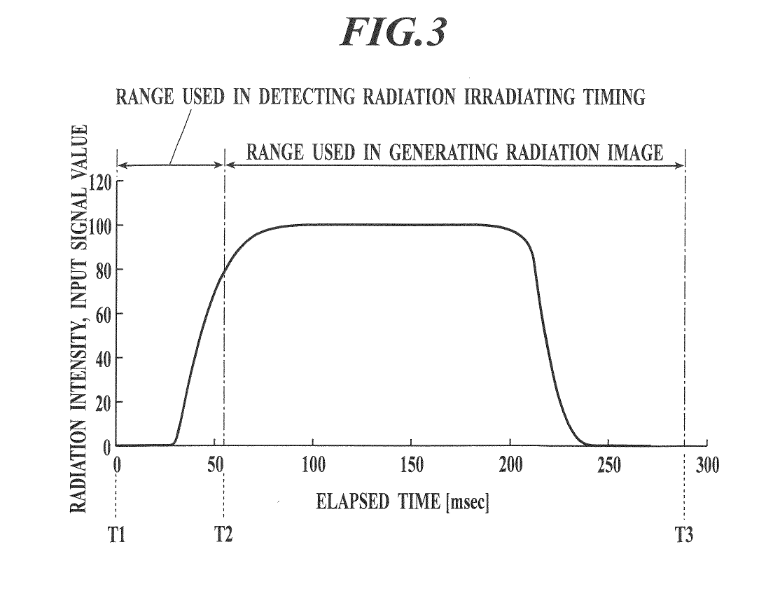

The method of detecting the irradiating timing of the radiation is described with reference to FIG. 3. A horizontal axis shows amount of time which passes (msec), and a vertical axis shows intensity (input signal value) of the radiation which the radiation detecting elements 121 receive for each unit area. The control section 11 monitors the radiation detecting element 121 of the detector 12 as a target of detecting irradiating timing to see whether the radiation intensity exceeds a predetermined threshold from a time point T1. When the radiation intensity exceeds the threshold (time point T2), it is judged that the radiation is irradiated. Then, the control section 11 controls the start of accumulating charge for generating the radiation image at the time point T2 when the radiation intensity exceeds the threshold. The control section 11 reads the charge at the time point T3 after a predetermined amount of time passes from the time point T2, and generates the image data.

According to the present embodiment, in addition to the radiation intensity, an integrated value integrating the radiation intensity of the latest certain amount of time (for example, 50 msec, etc.) (from a certain amount of time before to the present) is also used. When the integrated value of the radiation intensity exceeds a predetermined threshold, the control section 11 judges that the radiation is irradiated.

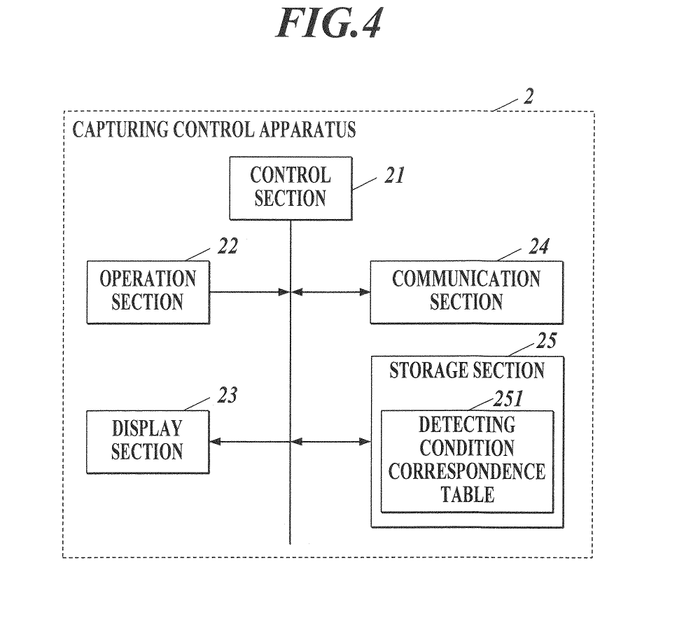

FIG. 4 shows a functional configuration of the capturing control apparatus 2. As shown in FIG. 4, the capturing control apparatus 2 includes a control section 21, an operation section 22, a display section 23, a communication section 24, a storage section 25 and the like.

The control section 21 includes a CPU, a RAM, and the like. The CPU of the control section 21 reads various programs such as a system program, processing program, etc., stored in the storage section 25 and expands the program in the RAM. The control section 21 performs various processing according to the expanded program.

The operation section 22 includes a keyboard including character input keys, numeral input keys, various function keys, etc., and a pointing device such as a mouse, etc. The pressed signal of the key pressed on the keyboard and the operation signal by the mouse are output as input signals to the control section 21. For example, the operation section 22 is used when the change of detecting condition is allowed.

The display section 23 includes a monitor such as an LCD (Liquid Crystal Display), etc., and displays various screens according to an instruction of a display signal input from the control section. For example, the display section 23 displays a warning that the detecting condition is changed.

It is possible to form a touch panel (not shown) of a pressure sensitive type (pressure sensitive resistance film type) in which transparent electrodes are positioned in a grid shape on the screen of the display section 23 to form a touch screen in which the display section 23 and the operation section 22 are configured as one. In this case, the touch panel detects the XY coordinate of a pressure point pressed by a finger, a stylus pen, etc., with the voltage value and outputs the detected position signal as the operation signal to the control section 21.

The communication section 24 transmits and receives data between the FPD panel 1 through the access point 4. The communication section 24 includes a network interface, etc. and transmits and receives data between external devices connected to the network.

The storage section 25 includes, for example, an HDD (Hard Disk Drive), a non-volatile semiconductor memory, and the like. The storage section 25 stores various programs, data necessary for various processing, etc. Specifically, the storage section 25 stores capturing order information regarding capturing with the FPD panel 1. The storage section 25 also stores in advance a detecting condition correspondence table 251, in which the capturing order information is corresponded with the detecting condition of irradiating timing of the radiation in the FPD panel 1.

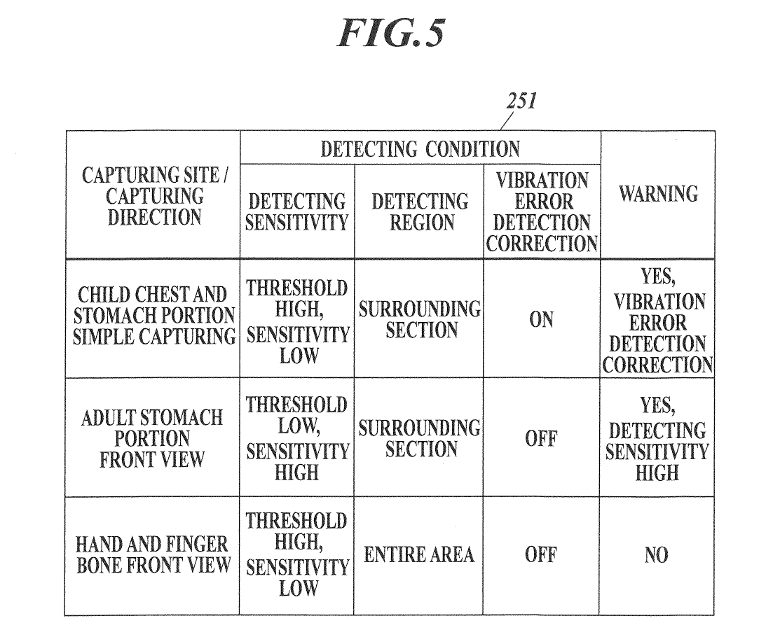

FIG. 5 shows an example of the detecting condition correspondence table 251. The detecting condition correspondence table 251 stores “capturing site/capturing direction”, “detecting condition”, and “warning” corresponded to each other. In the present embodiment, the capturing site and the capturing direction are used as the capturing order information used when deciding the detecting condition. The detecting condition includes “detecting sensitivity”, “detecting region”, and “vibration error detection correction”.

The “detecting sensitivity” is the sensitivity of the radiation detecting element 121 used when detecting the irradiating timing of the radiation. Specifically, the threshold of the radiation intensity or the threshold of the integrated value of the radiation intensity is stored corresponded to each sensitivity. The sensitivity can be changed by changing the threshold of the radiation sensitivity and the threshold of the integrated value of the radiation sensitivity. When the radiation intensity or the integrated value of the radiation intensity exceeds the threshold, it is judged that the radiation is irradiated from the radiation generating apparatus 5. For example, it is possible to prevent error in detection by raising the threshold (lowering the sensitivity). Alternatively, it is possible to prevent omission of detection by lowering the threshold (raising the sensitivity).

The “detecting region” is the region of the radiation detecting element 121 used when detecting the irradiating timing of the radiation. Specifically, the “detecting region” specifies the region which includes the radiation detecting element 121 to be used on the radiation entering surface of the FPD panel 1 among the radiation detecting elements 121 composing the FPD panel 1.

The “vibration error detection correction” is processing which does not consider sudden change of the signal value showing the radiation intensity. It is judged whether the change is sudden by, for example, whether the absolute value of the change of the signal value within a predetermined amount of time is equal to or more than a predetermined value. When the vibration error detection correction is ON, the vibration error detection correction is performed, and when the vibration error detection correction is OFF, the vibration error detection correction is not performed.

The “warning” is information showing whether to warn the user regarding the change of the detecting condition of the irradiating timing. When the warning is shown, the content of the change (content of the warning) in the detecting condition which is warned is included in the “warning” field of the detecting condition correspondence table 251.

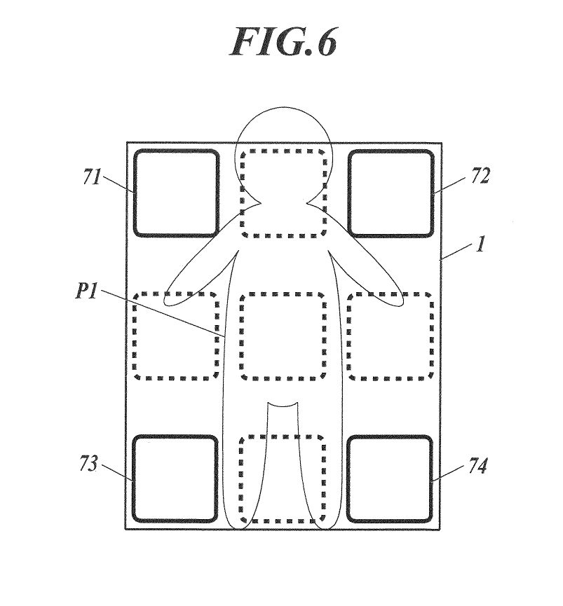

For example, in child chest and stomach portion simple capturing (front view), there are characteristics such as a high possibility that the subject P1 moves and causes vibration, and the amount of radiation which reaches the detector 12 becomes high. Therefore, the threshold in the detecting sensitivity can be set to a high value (lower the sensitivity) to prevent error in detection. As shown in FIG. 6, the detecting region is to be the surrounding section of the FPD panel 1 (regions 71 to 74), so that it is possible to detect the radiation in a region which does not overlap with the subject P1. A warning to turn ON the vibration error detection correction is displayed, and when the change of the detecting condition which is to turn ON the vibration error detection correction is allowed, the vibration error detection correction is turned ON. The vibration error detection correction can prevent error in detection due to vibration.

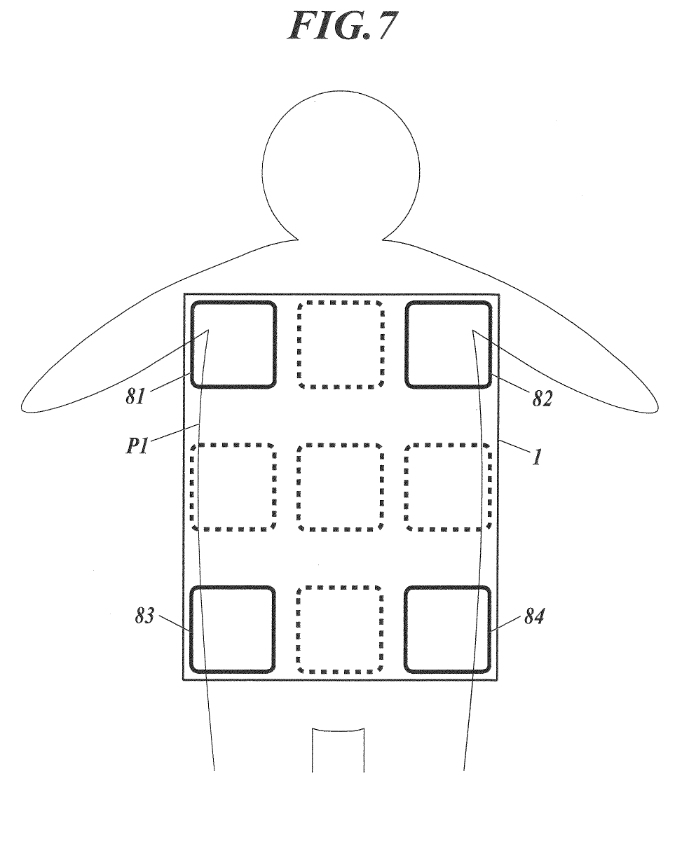

In adult stomach portion front view capturing, there are characteristics such as the vibration is small and when the person is fat, the amount of radiation which reaches the detector 12 is small. Therefore, the threshold of detection in the detecting sensitivity can be lowered (raise the sensitivity) to prevent omission of detection. As shown in FIG. 7, the detecting region can be set to the surrounding section of the FPD panel 1 (regions 81 to 84) so that it is possible to detect the radiation in a region where it is considered that the amount of radiation which reaches the detector 12 becomes comparatively large. A warning to raise the detecting sensitivity is displayed, and when the change in detecting condition which is to raise the detecting sensitivity is allowed, the detecting sensitivity is raised.

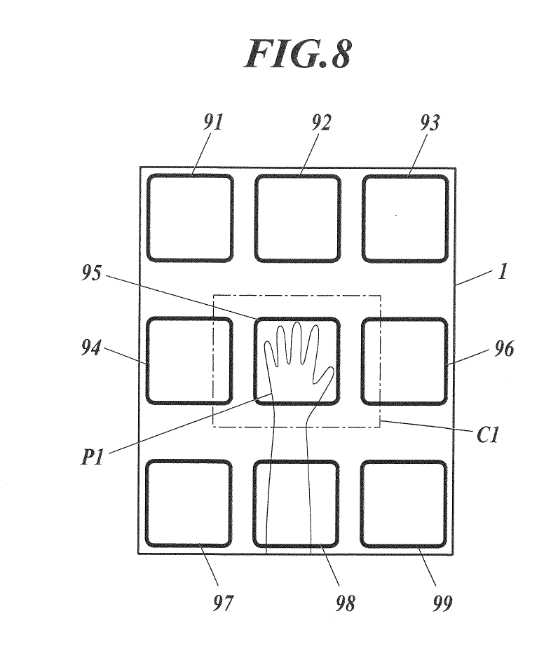

In hand and finger bone front view capturing, there are characteristics such as the vibration is small and the amount of radiation which reaches the detector 12 is large. Therefore, the threshold of detection in the detecting sensitivity can be raised (lower the sensitivity) to prevent error in detection. As shown in FIG. 8, the detecting region is to be the entire FPD panel 1 (regions 91 to 99). In hand and finger bone front view capturing, the irradiating field C1 is narrowed with respect to the subject P1. Therefore, if the detecting region is only the surrounding section, there is a possibility of omission of detection. In hand and finger bone front view capturing, a warning is not displayed regarding changing the detecting condition of the irradiating timing.

The control section 21 obtains the capturing order information regarding capturing with the FPD panel 1 from the RIS through the communication section 24. In other words, the control section 21 functions as the obtaining section.

The control section 21 reads the detecting condition corresponded to the obtained capturing order information (capturing site/capturing direction) from the detecting condition correspondence table 251 of the storage section 25, and transmits the read detecting condition to the FPD panel 1 through the communication section 24. In other words, the control section 21 functions as the first control section.

When the “warning” corresponded to the capturing site/capturing direction included in the capturing order information is “YES” in the detecting condition correspondence table 251 of the storage section 25, the control section 21 displays the warning regarding the specified detecting condition in the “warning” field on the display section 23.

When the change of the detecting condition which is warned is allowed by the operation on the operation section 22, the control section 21 transmits the detecting condition in which the change is allowed to the FPD panel 1. Specifically, when the change of the detecting condition which is warned is allowed by the operation on the operation section 22, the control section 21 transmits the detecting condition corresponded to the capturing site/capturing direction including the detecting condition which is warned to the FPD panel 1. Alternatively, when the change of the detecting condition which is warned is not allowed by the operation on the operation section 22, the control section 21 transmits the detecting condition corresponded to the capturing site/capturing direction excluding the detecting condition which is warned to the FPD panel 1.

### [Operation of the Radiation Image Capturing System]

Next, the operation of the FPD panel 1 and the capturing control apparatus 2 is described.

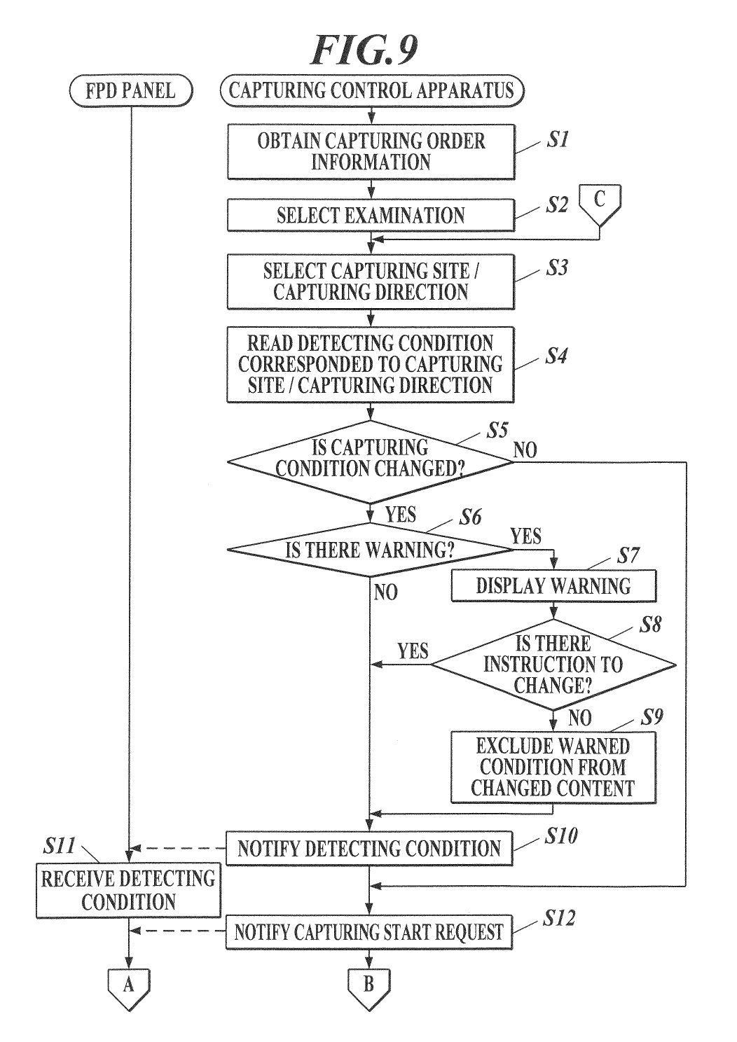

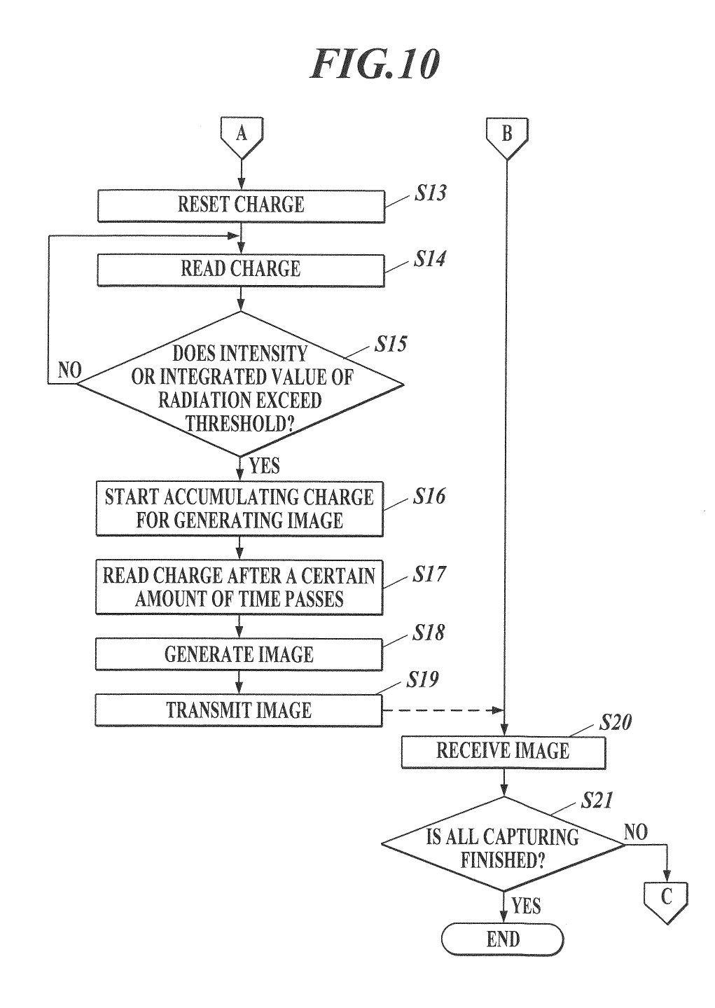

FIG. 9 and FIG. 10 are ladder charts showing radiation image capturing processing with the FPD panel 1 and the capturing control apparatus 2.

First, in the capturing control apparatus 2, the control section 21 obtains the capturing order information from the RIS through the communication section 24 (step S1). The user can operate the operation section 22 to input the capturing order information.

Next, the control section 21 displays the list of examinations on the display section 23 based on the capturing order information. Then, the examination to be performed is selected from the list of examinations by operation on the operation section 22 (step S2).

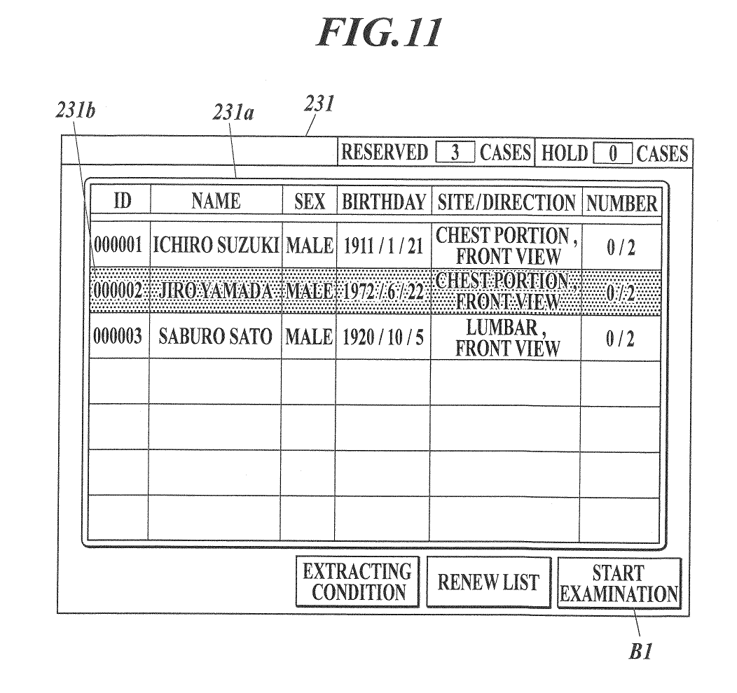

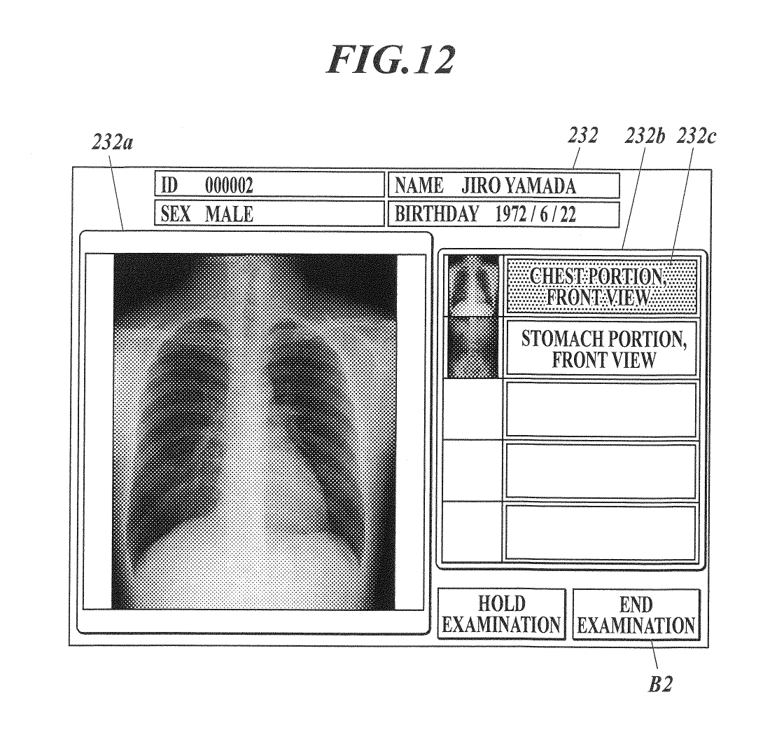

FIG. 11 shows an example of a list screen 231 displayed on the display section 23. A patient ID, a patient name, sex, birthday, capturing site/capturing direction and number of images are displayed for each examination in an examination list display region 231a of the list screen 231. An examination 231b is selected from the examination list display region 231a, and when an examination start button B1 is pressed, the screen advances to the capturing screen 232 (see FIG. 12).

Next, the control section 21 displays the capturing site/capturing direction included in the selected examination based on the capturing order information corresponding to the selected examination on the display section 23. Then, the capturing site/capturing direction to be executed is selected by operation on the operation section 22 from the displayed capturing site/capturing direction (step S3).

FIG. 12 shows an example of a capturing screen 232. A captured image display region 232a and a site/direction display region 232b are included in the capturing screen 232. An image is not displayed in the captured image display region 232a before capturing, and the captured image is displayed after capturing. When a capturing site/capturing direction 232c is selected from the site/direction display region 232b, the capturing of the selected capturing site is performed in the selected capturing direction.

Next, the control section 21 refers to the detecting condition correspondence table 251 stored in the storage section 25 based on the selected capturing site/capturing direction, and the control section 21 reads the detecting condition (detecting sensitivity, detecting region, vibration error detection correction) of the irradiating timing of the radiation corresponded to the selected capturing site/capturing direction (step S4).

Next, the control section 21 compares the read detecting condition of the irradiating timing and the presently set detecting condition of the irradiating timing and judges whether to change the detecting condition of the irradiating timing (step S5).

When the read detecting condition of the irradiating timing and the presently set detecting condition of the irradiating timing are different, in other words, when the detecting condition of the irradiating timing is changed (step S5; YES), the control section 21 judges whether the content to be warned is included in the changed content (step S6). Specifically, when the “warning” field of the detecting condition correspondence table 251 shows “YES”, it is judged that the content to be warned is included and when the “warning” field shows “NO”, it is judged that the content to be warned is not included.

When the content to be warned is not included in the changed content (step S6; NO), the entire content of the detecting condition of the irradiating timing read in step S4 is considered to be changed and the processing advances to step S10.

In step S6, when the content to be warned is included in the changed content (step S6; YES), the control section 21 displays a warning on the display section 23 regarding the detecting condition specified in the “warning” field of the detecting condition correspondence table 251 (step S7).

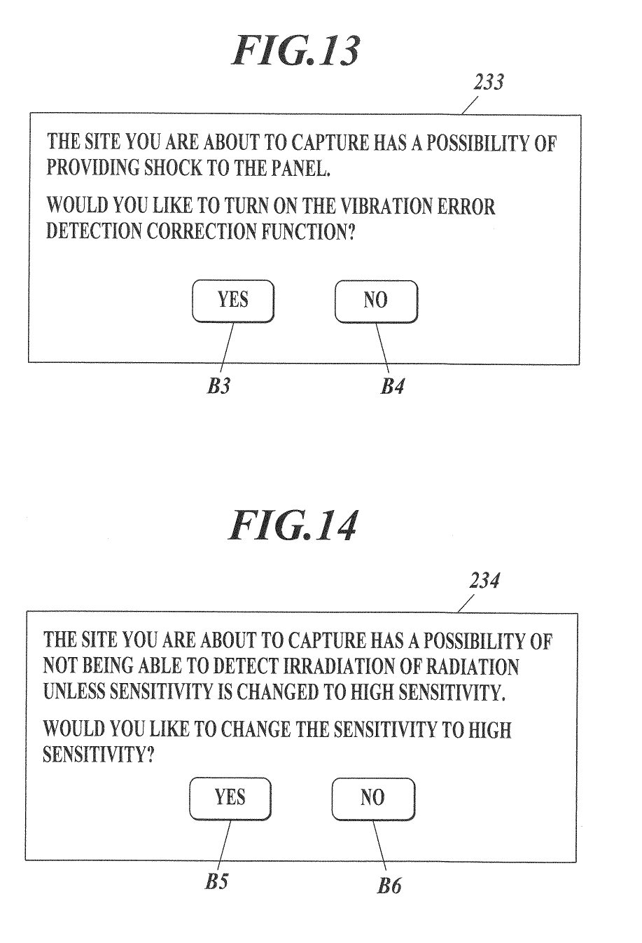

FIG. 13 shows an example of the warning message screen 233 to change the vibration error detection correction function to ON. The warning message screen 233 includes a YES button B3 to instruct that change to turn ON the vibration error detection correction function is allowed, and a NO button B4 to instruct that change to turn ON the vibration error detection correction function is not allowed.

FIG. 14 shows an example of a warning message screen 234 to change the detecting sensitivity to a high sensitivity. The warning message screen 234 includes a YES button B5 to instruct that the change of the detecting sensitivity to high sensitivity is allowed and a NO button B6 to instruct that the change of the detecting sensitivity to high sensitivity is not allowed.

When the instruction to allow the change of the detecting condition shown in the warning is input by operation of the operation section 22 (step S8; YES), the entire content of the detecting condition of the irradiating timing read in step S4 including the detecting condition shown in the warning is considered to be changed and the processing advances to step S10.

For example, when the YES button B3 is pressed in the warning message screen 233 shown in FIG. 13, or the YES button B5 is pressed in the warning message screen 234 shown in FIG. 14, the control section 21 judges that there is an instruction to allow change of the detecting condition displayed in the warning.

In step S8, when the instruction to not allow the change of the detecting condition shown in the warning is input by operation of the operation section 22 (step S8; NO), the control section 21 removes the detecting condition shown in the warning from the changed content (step S9), and the processing advances to step S10.

For example, when the NO button B4 is pressed in the warning message screen 233 shown in FIG. 13, or the NO button B6 is pressed in the warning message screen 234 shown in FIG. 14, the control section 21 judges that there is an instruction to not allow change of the detecting condition shown in the warning.

When the content to be warned is not included in the changed content in step S6 (step S6; NO), when the instruction to allow the change of the detecting condition shown in the warning is input in step S8 (step S8; YES), or after step S9, the control section 21 notifies the detecting condition of the irradiating timing through the communication section 24 to the FPD panel 1 (step S10).

In the FPD panel 1, the wireless communication section 13 receives the detecting condition from the capturing control apparatus 2 through the access point 4 (step S11). The control section 11 stores the received detecting condition, in other words, the changed detecting condition of the irradiating timing in the storage section 14.

In the capturing control apparatus 2, after step S10 or when the detecting condition of the irradiating timing is not changed in step S5 (step S5; NO), once the preparation for capturing is completed, the control section 21 notifies a capturing start request to the FPD panel 1 through the communication section 24 based on operation of the operation section 22 (step S12).

Advancing to FIG. 10, in the FPD panel 1, when the capturing start request is received by the wireless communication section 13 from the capturing control apparatus 2 through the access point 4, the control section 11 resets the charge of the radiation detecting elements 121 of the detector 12 (step S13).

Next, the control section 11 reads the charge of the radiation detecting elements 121 of the detector 12 (step S14), and judges whether the radiation intensity or the integrated value of the radiation intensity exceeds the set threshold based on the detecting condition of the irradiating timing of the radiation stored in the storage section 14 (step S15). When both the radiation intensity and the integrated value of the radiation intensity does not exceed the threshold (step S15; NO), the processing returns to step S14.

In step S15, when the radiation intensity or the integrated value of the radiation intensity exceeds the threshold (step S15; YES), the control section 11 judges that the radiation is irradiated from the radiation generating apparatus 5, and the radiation detecting elements 121 of the detector 12 start accumulating charge for generating the image (step S16). Next, after a certain amount of time passes, the control section 11 reads the charge of the radiation detecting elements 121 of the detector 12 (step S17), and generates the radiation image (step S18). The control section 11 stores the image data of the generated radiation image in the storage section 14. Next, the control section 11 transmits the generated radiation image to the capturing control apparatus 2 through the wireless communication section 13 (step S19).

In the capturing control apparatus 2, the communication section 24 receives the radiation image (step S20) and the control section 21 stores the received radiation image in the storage section 25.

Here, when there is capturing which is not finished (step S21; NO), the processing returns to step S3 in FIG. 9, and the processing is repeated.

In step S21, when all of the capturing is finished (step S21; YES), the radiation image capturing processing ends.

For example, when an examination end button B2 on the capturing screen 232 (see FIG. 12) is pressed by operation of the operation section 22, the examination ends.

As described above, according to the present embodiment, the timing of irradiating the radiation by the radiation generating apparatus 5 is detected based on the detecting condition corresponded to the capturing order information (capturing site/capturing direction). Therefore, it is possible to prevent error in detection and omission of detection when detecting the irradiating timing of the radiation with the FPD panel 1 itself.

For example, when the amount of radiation which reaches the detector 12 is large in capturing such as child chest and stomach portion simple capturing and hand and finger bone front view capturing, the sensitivity is set to a low value to be able to prevent error in detection. Moreover, when the amount of radiation which reaches the detector 12 is small in capturing such as adult stomach portion front view capturing, the sensitivity is set to a high value to be able to prevent omission of detection.

When the capturing is performed by narrowing the irradiating field in capturing such as hand and finger bone front view capturing, the detecting region is enlarged to the entire region to be able to prevent omission of detection.

When the subject P1 easily moves and shock is easily applied to the FPD panel 1 in capturing such as child chest and stomach portion system capturing, the vibration error detection correction is turned ON to prevent error in detection due to vibration.

In the capturing control apparatus 2, when the detecting condition is changed, a warning is displayed for the changed content which should be warned. Therefore, it is possible to draw attention to the user and to prevent change in detecting condition which is not desired by the user.

The description of the present embodiment is one example of the radiation image capturing system of the present invention, and the present invention is not limited to the above. The detailed configuration and the detailed operation of the devices composing the system can be changed without leaving the scope of the present invention.

For example, according to the present embodiment, the detecting condition of the irradiating timing of the radiation is changed based on the capturing site and the capturing direction included in the capturing order information. However, the detecting condition of the irradiating timing can be changed suitably based on only the capturing site or only the capturing direction. Moreover, the detecting condition of the irradiating timing can be changed suitably based on the age and sex included in the capturing order information.

According to the present embodiment, the detecting sensitivity and the detecting region are changed according to the capturing order information, however either one of the detecting sensitivity or the detecting region may be changed. The detecting condition other than the detecting sensitivity, the detecting region, and the ON and OFF of the vibration error detection correction may be changed.

The method of detecting the radiation irradiating timing on the FPD panel 1 is not limited to the above embodiment.

The present application is based on Japanese Patent Application No. 2012-269977 filed on Dec. 11, 2012 to the Japanese Patent Office, which shall be a basis for correcting mistranslations.

What is claimed is:

1. A radiation image capturing system comprising:
a radiation image capturing apparatus which is provided with a plurality of radiation detecting elements to convert radiation to an electric signal, wherein the radiation image capturing apparatus detects irradiating timing of radiation by a radiation generating apparatus and generates an image based on the radiation which passes through a subject when the radiation is irradiated; and
a capturing control apparatus which is connected to the radiation image capturing apparatus to be able to communicate data and which controls the radiation image capturing apparatus,
wherein the capturing control apparatus includes,
a storage section which stores capturing order information corresponded with a detecting condition of irradiating timing of radiation by the radiation image capturing apparatus;
an obtaining section which obtains capturing order information regarding capturing by the radiation image capturing apparatus; and
a first control section which reads the detecting condition corresponded to the obtained capturing order information from the storage section and which transmits the read detecting condition to the radiation image capturing apparatus, and
the radiation image capturing apparatus includes,
a second control section which detects the irradiating timing of the radiation by the radiation generating apparatus based on the detecting condition transmitted from the capturing control apparatus.

2. The radiation image capturing system of claim 1, wherein the detecting condition includes sensitivity and/or region of the plurality of radiation detecting elements used when detecting the irradiating timing of the radiation.

3. The radiation image capturing system of claim 1, wherein the capturing order information includes a capturing site and/or a capturing direction.

4. The radiation image capturing system of claim 1, wherein,
the capturing control apparatus includes,
a display section which displays a warning that the detecting condition is to be changed; and
an operation section to input an instruction to allow the detecting condition to be changed,
wherein when an instruction to allow the detecting condition to be changed is input on the operation section, the first control section transmits the detecting condition which is allowed to be changed to the radiation image capturing apparatus.
